(19) United States
(12) Patent Application Publication
(10) Pub. No.: US 2007/0131585 A1
(43) Pub. Date: Jun. 14, 2007

(54) High-speed chamber mixer for catalytic oil suspensions as a reactor for the depolymerization and polymerization of hydrocarbon-containing residues in the oil circulation to obtain middle distillate

(76) Inventor: Christian Koch, Buttenheim (DE)

Correspondence Address: COHEN, PONTANI, LIEBERMAN & PAVANE, 551 FIFTH AVENUE, SUITE 1210, NEW YORK, NY, 10176, US

(21) Appl. No.: 11/508,760

(22) Filed: Aug. 23, 2006

(30) Foreign Application Priority Data
Nov. 29, 2005 (DE) 10 2005 056 735.5

(51) Int. Cl. C07C 4/00

(52) U.S. Cl. 208/106; 202/82; 202/83; 366/219; 203/50; 585/240

(57) ABSTRACT

Production of diesel oil from hydrocarbon-containing residues in an oil circulation with separation of solids and product distillation for the diesel oil product by energy input with a high-speed chamber mixer and the use of fully crystallized catalysts that consist of potassium-, sodium-, calcium-, and magnesium-aluminum silicates, wherein the energy input and conversion occur primarily in the high-speed chamber mixer.

### BACKGROUND OF THE INVENTION

1. Field of the Invention

The invention relates to a method and an apparatus for the extraction of hydrocarbon vapor from residues in the temperature range of 230-380° C. in the hot oil circulation with a single-stage or multistage mixing chamber, which utilizes a pump with extremely low efficiency on the delivery side and the production of up to 95% vacuum on the intake side. In the process, the extracted hydrocarbons are depolymerized, deoxygenized, and freed of inorganic components of the molecule, such as halogens, sulfur, and heavy metal atoms.

2. Description of the Related Art

A depolymerization system with a hot oil circulation is known from German Patent No. 100 49 377 and German Published Patent Application No. 103 56 245.1. Here too, ion-exchanging catalysts are used in the hot oil circulation. The heat of reaction is supplied by heat transfer through the wall or by conduction by a pump with frictional heat.

The disadvantage of the methods and devices disclosed in German Patent No. 100 49 377 is the excessive temperature at the wall during the heat transfer, which results in pyrolytic reactions, and with respect to those disclosed in German Patent Application No. 103 56 245.1, the short residence time in a pump of less than one second, which is insufficient for the reaction of the residue with the catalyst oil. The actual reaction must then take place in the downstream equipment, which is possible only at significantly higher temperatures than if the reaction could take place relatively completely with a longer residence time in the pump.

Other disadvantages are the high pressure that develops in the pump and that can lead to clogging in the downstream pipes, which are typically narrower, the possible cavitation in the pump intake zone, especially for substances that contain solids, and the possible clogging of the intake zone if suction intake is not possible with relatively high negative pressure.

### SUMMARY OF THE INVENTION

All of these disadvantages are now eliminated by the present invention which utilizes a high-speed chamber mixer. As a result, the quality of the process and the product and the safety of the plant are dramatically improved. In this regard, the use of a system with rolls for the suction of gases in the application for realizing a hot oil circulation is novel.

Specifically, only the principle of the liquid ring vacuum pump was previously known, according to which gases can be compressed to atmospheric pressure, and up to about 1.5 bars of overpressure can be used as a compressor. What was not known and what was surprisingly discovered is that this principle for the conveyance of liquids and liquid/gas mixtures can be used in a mixing reactor. By utilizing the extremely low efficiency and the production of mixing and frictional energy between the catalyst oil and the hydrocarbon-containing residue feed, this system is the ideal energy-transfer unit for the production of diesel oil from residues.

This basic principle thus represents only a framework, which becomes the high-speed chamber mixer of the present invention by virtue of the completely new design of the components for the load oil instead of gas. Thus, compared to the previous pumps in German Patent Application No. 103 56 245.1, an overpressure in the delivery line of 6-100 bars becomes a pressure load of 0.5-2.0 bars, and the maximum negative pressure in the intake line of 0.1 bar (to avoid cavitation) becomes a possible negative pressure of 0.95 bar, i.e., a 95% vacuum.

The high-speed chamber mixer with the connecting pipelines, the volume flow control valve and a separator form a hot oil circulation, which, with the action of the molecularly fine, 100% crystalline catalyst, extracts the hydrocarbons from the preheated and dewatered hydrocarbon-containing residues, and at the same time, depending on molecular length, the extracted hydrocarbons are depolymerized, polymerized, deoxygenized, and freed of their inorganic components, such as halogens, sulfur, and heavy metal atoms. The product results from the reaction temperature of 250-320° C. in the middle distillate range, i.e., diesel fuel for use in diesel engines.

The basis of this process is the possible fast reaction process with intensive energy input with sufficient residence time, as is possible only in a high-speed chamber mixer. Pumping systems achieve only a very small fraction of this residence time and thus do not achieve the necessary reaction conditions and the low reaction temperatures associated therewith. In this process the goal is to keep the interval between the pyrolysis temperature and the catalytic depolymerization temperature as large as possible, i.e., to achieve the lowest possible reaction temperature.

In this regard, measurements showed that the average temperature with the high-speed chamber mixer is 60° C. lower than with the same system but different conveyance systems, for example, a pumping system with centrifugal impellers. This results in a decisive improvement compared to previously known systems, such as the system described in German Patent Application No. 103 56 245.1, especially with respect to the quality and odor of the product that is produced.

The uniformity of the middle distillates that are produced, which is apparent in the compressed curve of the gas chromatogram, the reduced energy input, and, finally, in the completeness of the reaction, is significantly increased. The selectivity of the process increases significantly, i.e., the yield of middle distillate increases, and the fraction of separated carbon drops in the case of plant feedstocks. The fractions of light products (odorous substances) are almost completely avoided.

Other objects and features of the present invention will become apparent from the following detailed description considered in conjunction with the accompanying drawings. It is to be understood, however, that the drawings are intended solely for purposes of illustration and not as a definition of the limits of the invention, for which reference should be made to the appended claims.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference numerals delineate similar elements throughout the several views:

FIG. 1 is a schematic diagram of one embodiment of the present invention;

FIG. 2 is a schematic diagram of another embodiment of the present invention; and

FIG. 3 is a schematic diagram of the high-speed chamber mixer of the present invention.

### DETAILED DESCRIPTION OF THE PRESENTLY PREFERRED EMBODIMENTS

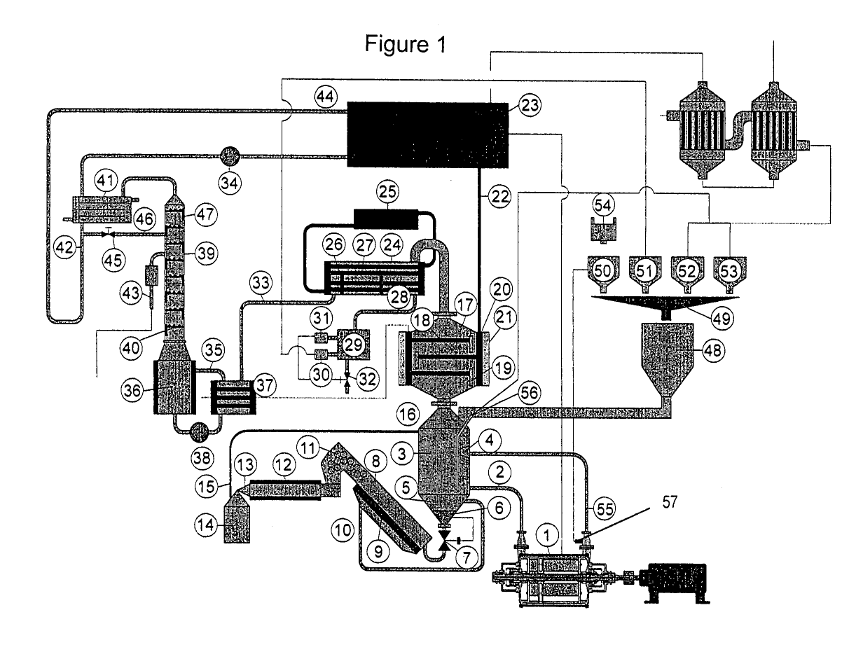

Referring to FIG. 1, a primary oil circulation is formed by a high-speed chamber mixer 1, its discharge outlet line 2 to a separator 3, and its intake line 55 from the separator 3. The separator 3 is a cyclone separator, which includes one or more venturi tubes 4, which are attached tangentially into the main body/cylindrical part of the separator 3 on the delivery side, and return lines located below in the cylindrical part. The conical part 5 located below collects deposits of residue slurry 6, which include inorganic constituents.

A pressure of 0.5-2.0 bars overpressure is obtained on the delivery side, depending on the size of the high-speed chamber mixer 1, and a pressure of 0.9-0.05 bar absolute, i.e., a 10% to 95% vacuum, is obtained on the intake side, depending on the solids content of residue slurry 6. An automatically controlled discharge valve 7 is installed below the separator 3, i.e., below the conical part 5. This discharge valve 7 opens as a function of the temperature, i.e., as a function of the fraction of the inorganic constituents of the material deposited there and thus allows the residue slurry 6 with the inorganic constituents to flow off into a pressure worm 8. The pressure worm 8 has a filter wall 9, through which the oil constituent of the residue slurry 6 is returned to the separator 3 via a return line 10, and thus forms a residue cake 11 towards the top, which enters a second conveyance device 12 with external heating. This conveyance device 12 has a nozzle 13 at its end, by which the inorganic solid residue, heated to 400-500° C., enters a storage tank 14, which has a connecting line 15 to the separator 3. The evaporated middle distillates 16 are returned to the process through this connecting line 15.

A vapor tank 17 is located above the separator 3. The purification elements of the vapor tank 17 include one or more distillation trays 18 with a reflux channel 19, a heater 20 and insulation 21 around the vapor tank 17, into which preferably exhaust gas 22 from a power generator 23 is introduced. This vapor tank 17 is connected with a condenser 24, which is cooled with cooling water from a cooling circulation 25. The condenser 24 has partition plates 26.

This results in the formation of chambers with overflows 27 to allow water to settle. In the front part, these chambers are connected by a line 28 with a water and pH tank 29, which has a pH meter 30 for measuring the pH, a conductivity cell 31 arranged above the pH meter 30, and a drain valve 32. The amount of water in the water tank 29 is automatically controlled by the drain valve 32 as a function of the water level determined by the conductivity cell 31.

A pipeline 33, which allows the condensate of the condenser 24 to be drained into a distillation system 39, is installed in the rear part of the condenser 24. The distillation system 39 includes a heat transfer medium circulation 37 between a circulation evaporator 36 of the distillation system 39 and an exhaust gas heat exchanger 41 of the power generator 23. The transfer medium circulation 37 has a connecting pipeline 35 to the distillation system 39, and a circulation pump 38. A vacuum pump 34 operates on the line connecting the power generator 23 with a second condenser 41. The distillation condenser 24 has a plurality of bubble trays 40. The distillation system 39 has a product discharge 43, and the second condenser 41 has a product discharge 42.

The product discharge 42 from the second condenser 41 serves as the fuel supply of the power generator 23 via a line 44, and a reflux valve 45 serves to feed the product reflux 46 into an upper distillation tray 47 in the distillation system 39. The product discharge 43 from upper column trays 47 of the distillation system 39 discharges the final product. This fraction generally contains 70-90% of the total amount of product.

The product that is removed is replaced by the addition of feedstock in a feed section 48. The feed section 48 includes a feed hopper 49 with a metering device 50 for catalyst, a metering device 51 for neutralizing agent (lime or soda), a liquid residue feed 52, and a solid residue feed 53.

The metering device 50 for the catalyst is usually connected with a big-bag emptying device 54, and the metering device 50 is controlled by a temperature measuring device 57 after the high-speed chamber mixer 1. If the heat transferred in the high-speed chamber mixer 1 is not sufficiently converted to the middle distillate product, and if the temperature rises above a limit, then the addition of catalyst in the metering device 50 increases.

The metering device 51 for the neutralizing agent is controlled by the pH meter 30. If the pH falls below an input limit of around 7.5, the feed amount in the metering device 51 increases. The added amounts of feed residues 52 and 53 are likewise metered as a function of a level gage 56 in the separator 3.

This ensures that the high-speed chamber mixer 1 always receives liquid mixtures from the separator 3 and that the system is prevented from running dry. It also ensures that the various feed materials and the associated variation of the reaction rates are always compensated by variable addition, and the process is not interrupted.

About 0.4 kWh of power for the cracking, evaporation, and heating from the input temperature of 250° C. to the reaction temperature of 300° C. are needed in the oil circulation per kg of evaporated diesel oil in the case of waste oil and tars. If plastics are used as feed materials, the required power is almost twice as high, since these materials are fed in cold, and energy for melting is additionally required.

In this regard, the addition of the catalyst is fundamentally important to the process. This catalyst is a sodium-aluminum silicate. The doping of a fully crystallized Y-molecule with sodium was determined to be optimum only for the plastics, bitumen, and waste oils. For biological feedstocks, such as fats and biological oils, doping with calcium was found to be optimum. For reactions with wood, doping with magnesium is necessary to produce high-quality diesel oil. For highly halogenated compounds, such as transformer oil and PVC, it is necessary to dope with potassium.

The product of the system is diesel oil, because the product discharge from the circulation at 300-400° C. leaves no other, lighter products behind in the system. 10% of this product is used to generate the process energy requirements in the form of electric current in a power-generating unit, and the portion used to generate power is the lighter fraction of the product obtained from the condenser.

The product from the column thus does not have a lighter boiling fraction and completely satisfies the tank storage standards. Another advantage of this energy conversion is the simultaneous solution of the problems with the gas emerging from the vacuum pump 34, which is conveyed into the intake air.

In addition, the power generator 23 satisfies the conditions of the combined heat and power generation, since the thermal energy of the exhaust gases is used to predry and preheat the feedstock.

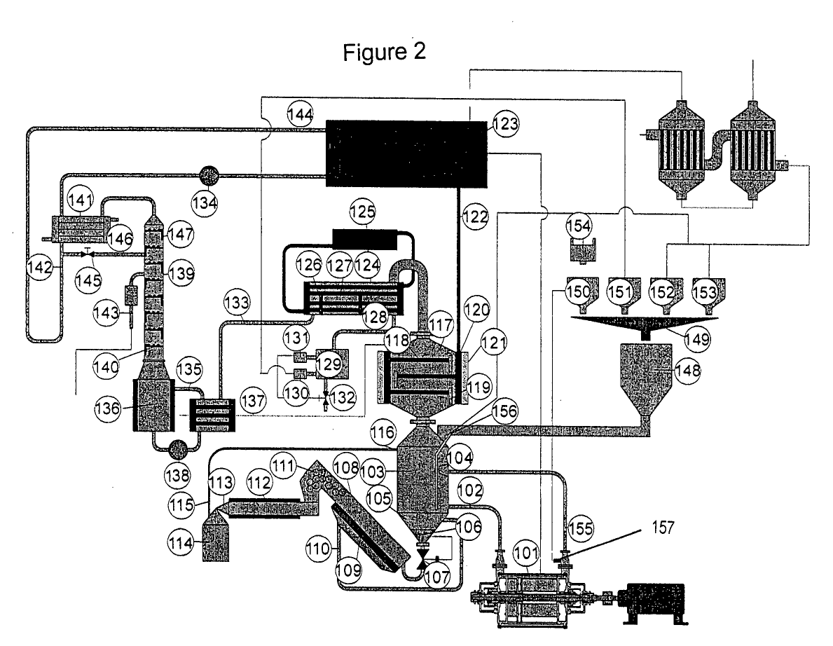

The device of the invention is further explained with reference to FIG. 2.

A high-speed chamber mixer 101 has an output line 102 connected to a separator 103 by a pipeline. Line 102 is designed for a negative pressure of 0.95 bar. The separator 103 is a cyclone separator, which includes one or more venturi tubes 104, which are attached tangentially into the main body/cylindrical part of the separator on the delivery side, and return lines located below in the cylindrical part.

The conical part 105 located below there has a discharge orifice 106 with an automatically controlled discharge valve 107. A pressure line 157 that is designed for an overpressure of 0.5-1.5 bars is arranged on the delivery side of the high-speed chamber mixer 101. The discharge valve 107 is installed below the separator 103, i.e., below the conical part 105. This discharge valve 107 has a temperature sensor, which is designed for a switching temperature of 100-150° C.

A pressure worm 108 is arranged below there, which is designed to convey residue slurry from the discharge valve 107 and has a temperature resistance of 200° C. The pressure worm 108 has a filter wall 109 with an oil outlet 110 and an upper pressure worm part for the residue cake 111 and a connecting pipeline to a second conveyance device 112 with external heating.

This conveyance device 112 has a nozzle 113 at the end. The worm wall is designed for a temperature of 400-500° C., which is produced by the external heater, e.g., an electric heater. A downstream storage tank 114 also has temperature resistance up to 400° C. and is designed as a solids tank. The storage tank 114 has a connecting line 115 to the separator 103 for returning the evaporated hydrocarbon vapor.

A vapor tank 117 is located above the separator 103. The purification elements of the vapor tank 117 include one or more distillation trays 118 with a reflux channel 119, a heater 120 and insulation 121 around the vapor tank 117, with an exhaust gas connecting line 122 to a power generator 123, by which exhaust gas is introduced into the vapor tank 117. This vapor tank 117 is connected with a condenser 124. The condenser 124 has a connecting line by which it receives cooling water from a cooling circulation 125. The condenser 124 has partition plates 126.

This results in the formation of chambers with overflows 127. In the front part, these chambers are connected by a line 128 with a water and pH tank 129, which has a pH meter 130 for measuring the pH, a conductivity cell 131 arranged above the pH meter 130, and a drain valve 132. The water level measurement in the water and pH tank 129 by conductivity measurement automatically controls the drain valve 132 as a function of the water level measured by the conductivity cell 131.

A pipeline 133, which allows the condensate of the condenser 124 to be drained into a distillation system 139, is installed in the rear part of the condenser 124. The distillation system 139 includes a heat transfer medium circulation 137 between a circulation evaporator 136 of the distillation system 139 and an exhaust gas heat exchanger 141 of the power generator 123. The transfer medium circulation 137 has a connecting pipeline 135 to the distillation system 139 and a circulation pump 138. A vacuum pump 134 operates on the line connecting the power generator 123 with a second condenser 141. The distillation condenser 124 has a plurality of bubble trays. The distillation system 139 has a product discharge 143, and the second condenser has a product discharge 142.

The product discharge 142 from the second condenser 141 has a connecting line 144 to a fuel supply tank of the power generator 123, and a reflux valve 145 serves to feed the product reflux 146 into an upper distillation tray 147 in the distillation system 139. The product discharge 143 from upper column trays 147 of the distillation system 139 discharges the final product. This line generally carries 70-90% of the total amount of product.

The device has an additional line for the addition of feedstock, which is located in a feed section 148. The feed section 148 includes a feed hopper 149 with a metering device 150 for catalyst, a metering device 151 for neutralizing agent (lime or soda), a liquid residue feed 152, and a solid residue feed 153.

The metering device 150 for the catalyst is usually connected with a big-bag emptying device 154, and the metering device 150 is controlled by a temperature measuring device 157 after the high-speed chamber mixer 101. If the heat transferred in the high-speed chamber mixer 101 is not sufficiently converted to the middle distillate product, and if the temperature rises above a limit, then the addition of catalyst in the metering device 150 increases.

The metering device 151 for the neutralizing agent is controlled by the pH meter 130. If the pH falls below an input limit of around 7.5, the feed amount in the metering device 151 increases. The added amounts of feed residues 152 and 153 are likewise metered as a function of a level gage 156 in the separator 103.

This ensures that the high-speed chamber mixture 101 always receives liquid mixtures from the separator 103 and that the system is prevented from running dry. It also ensures that the various feed materials and the associated variation of the reaction rates are always compensated by variable addition, and the process is not interrupted.

About 0.4 kWh of power for the cracking, evaporation, and heating from the input temperature of 250° C. to the reaction temperature of 300° C. are needed in the oil circulation per kg of evaporated diesel oil in the case of waste oil and tars. If plastics are used as feed materials, the required power is almost twice as high, since these materials are fed in cold, and energy for melting is additionally required.

In this regard, the addition of the catalyst is fundamentally important to the process. This catalyst is a sodium-aluminum silicate. The doping of a fully crystallized Y-molecule with sodium was determined to be optimum only for the plastics, bitumen, and waste oils.

For biological feedstocks, such as fats and biological oils, doping with calcium was found to be optimum. For reactions with wood, doping with magnesium is necessary to produce high-quality diesel oil. For highly halogenated compounds, such as transformer oil and PVC, it is necessary to dope with potassium.

The product of the system is diesel oil, because the product discharge from the circulation at 300-400° C. leaves no other, lighter products behind in the system.

10% of this product is used to generate the process energy requirements in the form of electric current in a power-generating unit, and the portion used to generate power is the lighter fraction of the product obtained from the condenser.

The product from the column thus does not have a lighter boiling fraction and completely satisfies the tank storage standards. Another advantage of this energy conversion is the simultaneous solution of the problems with the gas emerging from the vacuum pump 134, which is conveyed into the intake air. In addition, the power generator 123 satisfies the conditions of the combined heat and power generation, since the thermal energy of the exhaust gases is used to predry and preheat the feedstock.

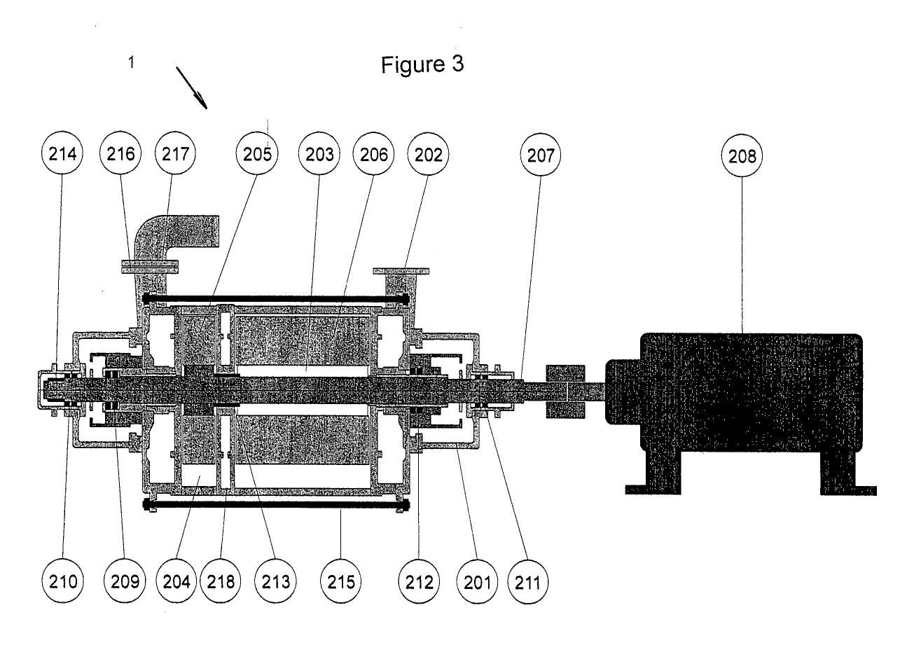

FIG. 3 shows the central unit of the method of the invention and the device of the invention, the high-speed chamber mixer 1. Reference numeral 201 denotes the housing of the high-speed chamber mixer. Reference numeral 202 denotes the intake (and is shown in FIG. 1 as element 55). Reference numerals 203 and 204 denote chambers contained in the high-speed chamber mixer 1. The chambers 203 and 204 have different sizes in the standard design and the same size in the special design. Roller wheels 205 and 206 run eccentrically in the chambers 204 and 203, respectively, and have three reinforcing ribs at the beginning, in the middle, and at the end.

The roller wheels 205 and 206 are driven by a shaft 207, which is connected at one end to an electric motor or diesel engine 208. The shaft 207 is supported by special bearings 209, 210, 211, 212 made of sintered hard metal in clamping rings. A ball bearing 213 and a sealing bearing 214 are mounted at the end of the shaft 207. The housing 201 is held together by tightening screws 215. A discharge outlet 216 (shown in FIG. 1 as element 2) is connected with a flange 217. A flow plate cam 218 is located between the two roller wheels 205 and 206 and has openings permitting fluid flow therethrough. The high-speed chamber mixer is sealed and has shaft bushings, with bellows seals or stuffing boxes or are realized without a packing by employing a magnetic coupling. The high-speed chamber mixer may have a connecting line from the bearings and seals to a cooling system. The roller wheels 205, 206 may be curved forwards or backwards. The roller wheels 205, 206 may be curved cylindrically or spatially.

The invention is explained in greater detail with reference to a specific embodiment. A high-speed chamber mixer with 120 kW of drive power conveys 2,000 L/h of intake oil through an intake line 2 and 300 kg of residual material in the form of waste oil and bitumen through the feed section 48 for a total of 2,300 L/h, into the delivery line, which opens tangentially into the separator 3 with a diameter of 800 mm.

The high-speed chamber mixer 1 is connected with the separator 3 by a connecting pipeline 55 with a diameter of 200 mm. An automatically controlled discharge valve 7, which controls the pressure in the downstream apparatus.

The separator 3 has a diameter of 1,000 mm, and on the inside it has a venturi tube 4, which has a cross section at its narrowest point of 100×200 mm, lies against the inside wall, and likewise decreases the remaining overpressure and increases the separation effect. Above the separator 3, there is a vapor tank 17 with a diameter of 2,000 mm. The separator 3 has a level control device 56, e.g., with an oil level gage.

The product vapor line for the diesel oil vapor that is produced is located at the top of the vapor tank 17 and runs to the condenser 24, which has a capacity of 100 kW. A line 33 with a diameter of 1.5 inches runs from the condenser 24 to the distillation system 39 with a column diameter of 300 mm. All of the tanks are provided with flue gas external heating to facilitate the heatup phase.

The pressure worm 8 with a diameter of 250 mm is located below the separator 3. It provides for the separation of the constituents of the feedstocks that cannot be converted to diesel oil. The pressure worm 8 is connected with the reducing pipe and discharge valve 7 with a diameter of 80 mm. A temperature measuring device is located at the base of the separator 3 and starts the operation of the pressure worm 8 when the temperature drops below a limit due to insulation by the residue.

The pressure worm 8, which has a diameter of 250 mm and a conveying capacity of 10-20 kg/h, has a filter wall 9 inside, which allows the liquid fractions to flow back into the separator 3, and an electrically heated low-temperature carbonization nozzle 13 at the end of the pressure worm 8 with a heating capacity of 45 kW, which allows the residual oil fractions to evaporate from the press cake 11. An increase in temperature to 500° C. is provided for this purpose. The oil vapors escaping from the low-temperature carbonization nozzle 13 are conveyed to the separator 3 through the return line 15.

Thus, while there have been shown and described and pointed out fundamental novel features of the present invention as applied to a preferred embodiment thereof, it will be understood that various omissions and substitutions and changes in the form and details of the devices described and illustrated, and in their operation, and of the methods described may be made by those skilled in the art without departing from the spirit of the present invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is also to be understood that the drawings are not necessarily drawn to scale but that they are merely conceptual in nature. It is the intention, therefore, to be limited only as indicated by the scope of the claims appended hereto.

What is claimed is:

1. A method for producing diesel oil from hydrocarbon-containing residues in an oil circulation with separation of solids and product distillation for the diesel oil product, comprising:
supplying heat by a high-speed chamber mixer.

2. The method of claim 1, wherein pump efficiency of the high-speed chamber mixer is low so that the energy input is primarily converted to mixing and frictional energy.

3. The method of claim 1, wherein the high-speed chamber mixer produces only a low overpressure of less than 2 bars on an output side and a possible high vacuum of up to 95% on an intake side.

4. The method of claim 1, wherein the high-speed chamber mixer produces and transfers lost energy to the oil circulation.

5. The method of claim 1, wherein the high-speed chamber mixer conveys pure or contaminated abrasive and chemically aggressive liquids.

6. The method of claim 1, wherein the high-speed chamber mixer produces vacuum and overpressure, is self-priming, and conveys liquids and liquid/gas mixtures.

7. The method of claim 1, wherein the high-speed chamber mixer can be operated as a stationary or mobile unit.

8. The method of claim 1, wherein a reaction in the high-speed chamber mixer is held to a conversion rate of 5-50% by a downstream valve.

9. The method of claim 1, further comprising automatically controlling a temperature level control system in the oil circulation by an automatic temperature control system and an automatic level control system which are linked with each other, and further comprising controlling a feed of hydrocarbon-containing residues and other constituents and controlling energy input so that a level in the oil circulation is maintained.

10. An apparatus for producing diesel oil from hydrocarbon-containing residues in an oil circulation with separation of solids and product distillation for the diesel oil product, comprising:
a separator with internal venturi tubes;
a high-speed chamber mixer in fluid connection to the separator;
a separation tank with heated discharge screw connected to the separator;
two product outlets downstream of the separator; and
two distillation systems, one at each of the two product outlets.

11. The apparatus of claim 10, wherein the high-speed chamber mixer comprises at least one roller wheel arranged centrically or eccentrically in at least one chamber of the high-speed chamber mixer.

12. The apparatus of claim 10, wherein the high-speed chamber mixer is arranged with an orientation between horizontal and vertical.

13. The apparatus of claim 10, wherein the high-speed chamber mixer is connectable to a prime mover by means of a coupling, and the direction of rotation can be set to left or right.

14. The apparatus of claim 10, wherein the high-speed chamber mixer has a single-stage or multistage mixing chamber, each chamber having different widths.

15. The apparatus of claim 10, wherein the high-speed chamber mixer has channels for draining residues.

16. The apparatus of claim 11, wherein the high-speed chamber mixer has two roller wheels and a flow plate cam positioned between the roller wheels with openings has openings therein permitting fluid flow therethrough.

17. The apparatus of claim 11, wherein the roller wheels are curved forwards or backwards.

18. The apparatus of claim 11, wherein the roller wheels are curved cylindrically or spatially.

19. The apparatus of claim 10, wherein the high-speed chamber mixer is sealed and further comprises shaft bushings, with bellows seals or stuffing boxes or are realized without a packing with a magnetic coupling.

20. The apparatus of claim 10, wherein the high-speed chamber comprises a shaft supported for rotation by bearings, and seals on the shaft, and wherein the high-speed chamber is connected at its bearings and seals to a cooling system.
